(19) United States
(12) Patent Application Publication
(10) Pub. No.: US 2019/0201620 A1
(43) Pub. Date: Jul. 4, 2019

(54) HIGH FLOW AT LOW PRESSURE INFUSION SYSTEM AND METHOD

(71) Applicant: REPRO-MED SYSTEMS, INC., Chester, NY (US)

(72) Inventor: Andrew I SEALFON, Monroe, NY (US)

(21) Appl. No.: 16/229,212

(22) Filed: Dec. 21, 2018

Related U.S. Application Data
(60) Provisional application No. 62/611,642, filed on Dec. 29, 2017.

(51) Int. Cl. A61M 5/168; A61M 5/145; A61M 5/32

(52) U.S. Cl. CPC A61M 5/16854; A61M 5/145; A61M 5/1413; A61M 5/16886; A61M 5/32

(57) ABSTRACT

Provided is a system and method for a high flow at low pressure infusion system needle set for delivering a liquid from a reservoir to a patient. More specifically, provided is a high flow at low pressure infusion system needle set including a flexible tubing element having a first length and a first end structured and arranged to connect to the reservoir, and a second end opposite thereto, the flexible tubing element having a first internal diameter. A needle having a second length and a second internal diameter is coupled to the second end of the flexible tubing, the needle having a first portion providing a sharpened distal end for penetration of the patient's tissue and a second portion providing a second end in fluid communication with the second end of the flexible tubing element, the first portion and second portion generally normal to each other. The second end of the needle has an outside diameter, the flexible tubing element having an average first internal diameter along the first length, the average first internal diameter at least 25% larger than the outside diameter of the second end of the needle. An associated method of use is also provided.

### CROSS REFERENCE TO RELATED APPLICATIONS

This application claims the benefit under 35 U.S.C. § 119(e) of U.S. Provisional Application No. 62/611,642 filed Dec. 29, 2017 and entitled HIGH FLOW AT LOW PRESSURE INFUSION SYSTEM AND METHOD, the disclosure of which is incorporated herein by reference.

### FIELD OF THE INVENTION

The present invention relates generally to systems and methods for liquid fluid flow as may be desired for the delivery of liquid for infusion to a patient, and more specifically to systems and methods to safeguard against overdose by providing a high flow rate at a low pressure.

### BACKGROUND

Infusion systems for the delivery of liquid pharmaceuticals are widely used and relied upon by patients and caregivers alike. Such delivery is generally made in one of two ways. The first is an immediate delivery from a health care provider or other operator in the form of a simple injection performed with a syringe and a needle directly disposed to the tissue of the patient.

For this type of immediate delivery, the amount of the pharmaceutical is typically measured by the health care provider or other operator and the rate of delivery is typically based on the speed at which they depress the plunger. Although overmedication can occur, the rate of delivery is rarely an issue with immediate delivery.

The second option is for gradual or prolonged delivery, wherein a syringe or other reservoir is connected to specific medical tubing for delivery over time. With such time-based delivery, overmedication and/or overdose of the pharmaceutical is a very real possibility. Syringes, or other pharmaceutical reservoirs such as fluid bags, are easily and commonly adapted for use with many different types of pharmaceutical, however the flow rate for proper delivery of such pharmaceuticals as determined by the manufacturer may vary widely. More specifically, a flow rate that is safe for infusion to a patient of one pharmaceutical, may not be appropriate for another, different pharmaceutical or patient.

In many cases, the pharmaceutical may be provided as a viscus liquid due to the nature of the compound to be administered. Compared to pure water, Sterile Water for Injection (SWFI) or Normal Saline, commonly referred to as an NS Infusion liquid, the viscosity of some pharmaceuticals can pose a challenge as it does not flow with the same properties as water or NS. Even with such viscus fluids, the rate of delivery is important so as to ensure that the patient receives proper treatment.

It has long been a standing belief within the infusion system market and community that the needle itself is the limiting factor for how fast, or how rapid the fluid flow rate would be for delivery into the patient's tissues.

But there are at least two issues of significant concern with rapid fluid delivery rate. Believing the needle to be the limiting factor, traditionally for rapid flow rate it has been the standard practice for subcutaneous administrations to use a larger needle, such as a 24-gauge needle. The larger the needle, the larger the trauma to the patient's tissues. So, while perhaps desirable for a rapid flow rate, from the patient's perspective a large gauge needle, such a 24-gauge, is likely more painful and less desirable then a smaller gauge needle, such as a 26-gauge needle.

As many infusion systems and/or treatment regiments may also employ multiple needles simultaneously, the use of multiple large gauge needles further escalates the patient's discomfort.

It has also been a common practice to use high-pressure electronic pump systems. While effective, such systems can be cost prohibitive for many users. In addition, most programmable electronic pumps are based on the principle of constant flow. Because these systems attempt to maintain the same flow rate regardless of pressure, these systems generally incorporate a warning system to alert the user and/or operator of any dangerous increase in pressure as the pump attempts to maintain that constant flow.

If there is an occlusion at the sight of administration, even with an alarm the patient may be injured and/or receive an overdose of the pharmaceutical. Indeed, in the effort to maintain a specific flow rate, constant flow pumps may inadvertently harm a patient by continuing to drive the fluid into a patient when his or her tissues are already saturated (for subcutaneous) or the vein is blocked (intravenous) or cannot otherwise receive the fluid at the provided rate.

Hence there is a need for a method and system that is capable of overcoming one or more of the above identified challenges.

### SUMMARY OF THE INVENTION

Our invention solves the problems of the prior art by providing a novel high flow at low pressure infusion system needle set system and method.

In particular, and by way of example only, according to one embodiment of the present invention, provided is a high flow at low pressure infusion system needle set for delivering a liquid from a reservoir to a patient, including: a flexible tubing element having a first length and a first end structured and arranged to connect to the reservoir, and a second end opposite thereto, the flexible tubing element having a first internal diameter; a needle having a second length and a second internal diameter, the needle having a first section providing a sharpened distal end for penetration of the patient's tissue and a second portion providing a second end in fluid communication with the second end of the flexible tubing element; wherein the second end of the needle has an outside diameter, the flexible tubing element having an average first internal diameter along the first length, the average first internal diameter at least 25% larger than the outside diameter of the second end of the needle.

For yet another embodiment, provided is a high flow at low pressure infusion system for delivering a liquid from a reservoir to a patient, including: a flexible tubing element having a first length of about 24″ and a first end structured and arranged to connect to the reservoir, and a second end opposite thereto, the tubing element having a first internal diameter of at least 0.33″; a needle having a maximum second length of about 0.95″ and a second internal diameter of about 0.0094″, the needle having a first section providing a sharpened distal end for penetration of the patient's tissue and a second portion providing a second end in fluid communication with the second end of the flexible tubing element, the first portion and second portion generally normal to each other; wherein the second end of the needle has an outside diameter, the flexible tubing element having an average first internal diameter along the first length, the average first internal diameter at least 25% larger than the outside diameter of the second end of the needle.

For still yet another embodiment, provided is a high flow at low pressure infusion system for delivering a liquid from a reservoir to a patient, including: a fluid pump for driving a fluid from the reservoir; a flexible tubing element having a first length and a first end structured and arranged to connect to the reservoir, and a second end opposite thereto, the tubing element having a first internal diameter selected with respect to the first length to provide laminar flow for a liquid having a known viscosity, received from the reservoir; a needle having a second length and a second internal diameter selected to maximize flow rate to a patient's tissues at a specific depth, the needle having a first section providing a sharpened distal end for penetration of the patient's tissue to the specific depth and a second portion providing a second end in fluid communication with the second end of the flexible tubing element, the first portion and second portion generally normal to each other; wherein the second end of the needle has an outside diameter, the flexible tubing element having an average first internal diameter along the first length, the average first internal diameter at least 25% larger than the outside diameter of the second end of the needle.

Still for yet another embodiment, provided is a method of using a high flow at low pressure infusion system needle set for delivering a liquid from a reservoir to a patient, comprising: coupling a reservoir containing a liquid to a pump for driving the liquid from the reservoir; providing a high flow at low pressure needle set including: a flexible tubing element having a first length and a first end structured and arranged to connect to the reservoir, and a second end opposite thereto, the flexible tubing element having a first internal diameter; a needle having a second length and a second internal diameter, the needle having a first portion providing a sharpened distal end for penetration of the patient's tissue and a second portion providing a second end in fluid communication with the second end of the flexible tubing element; wherein the second end of the needle has an outside diameter, the flexible tubing element having an average first internal diameter along the first length, the average first internal diameter at least 25% larger than the outside diameter of the second end of the needle; coupling the high flow at low pressure needle set to a reservoir and disposing the needle into a patient, and activating the pump.

### BRIEF DESCRIPTION OF THE DRAWINGS

FIGS. 1A, 1B and 1C are general illustrations of a high flow at low pressure infusion system in accordance with at least one embodiment;

FIG. 2 is an enlarged perspective view of the second end of the tubing and needle portions of the high flow at low pressure infusion system in accordance with at least one embodiment;

FIG. 3. Illustrates three versions for the needle element in accordance with varying embodiments of the present invention;

FIG. 4 is a conceptual system diagram of a high flow at low pressure infusion system in accordance with at least one embodiment;

FIG. 5 is a further general illustration of a high flow at low pressure infusion system in use in accordance with at least one embodiment;

FIG. 6 is a conceptual circuit diagram; and

FIG. 7 is a table of performance data for comparison of at least one embodiment of a high flow at low pressure infusion system in accordance with at least one embodiment.

### DETAILED DESCRIPTION

Before proceeding with the detailed description, it is to be appreciated that the present teaching is by way of example only, not by limitation. The concepts herein are not limited to use or application with a specific system or method for providing a high flow at low pressure system, needle set, or elements related thereto. Thus, although the instrumentalities described herein are for the convenience of explanation shown and described with respect to exemplary embodiments, it will be understood and appreciated that the principles herein may be applied equally in other types of high flow at low pressure infusions systems and method.

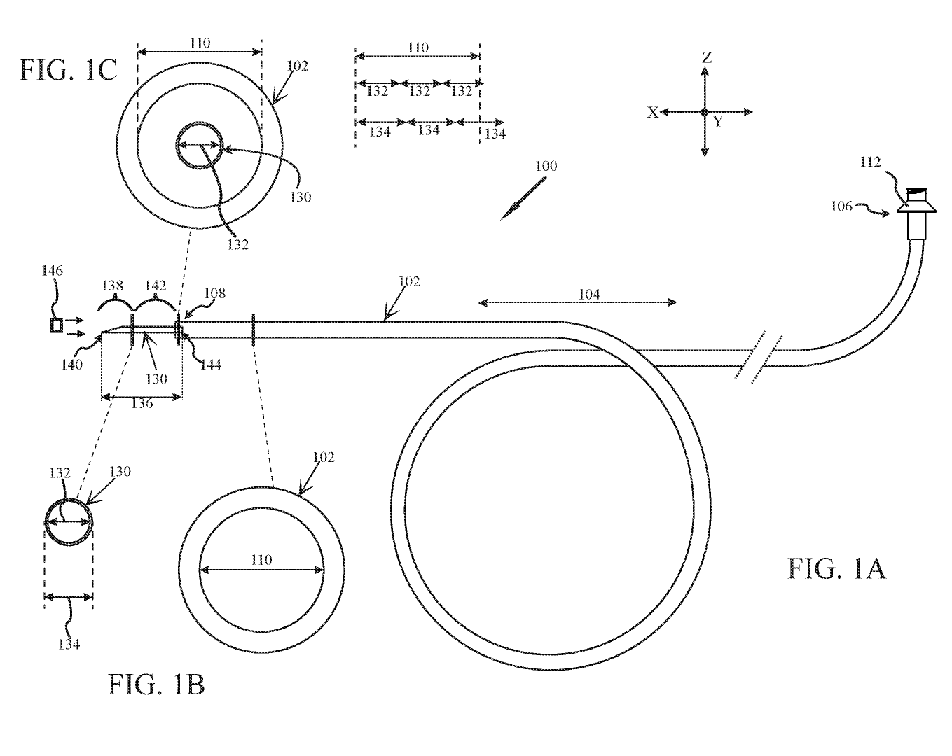

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 first appears in FIG. 1.

Turning now to FIG. 1, there is shown a high flow at low pressure infusion system needle set 100, hereinafter HFLPIS 100, in accordance with at least one embodiment of the present invention.

To facilitate the description of systems and methods for this HFLPIS 100, the orientation of HFLPIS 100, as presented in the figures, is referenced to the coordinate system with three axes orthogonal to one another as shown in FIG. 1. The axes intersect mutually at the origin of the coordinate system, which is chosen to be the center of HFLPIS 100, however the axes shown in all figures are offset from their actual locations for clarity and ease of illustration.

As shown, the HFLPIS 100 is comprised principally of a flexible tubing element 102, a needle 130, and a connector 112, such as a luer 112, which is more specifically a flared luer 112 in at least one embodiment as noted below.

Within the medical community, a needle set is understood and appreciated to be a device comprising several components—such as a connector 112 for attachment to a reservoir of a pharmaceutical or other liquid, a flexible tubing element 102 extending from the connector 112 to an assembly attaching the actual needle 130 to the flexible tubing element 102, through which the liquid will flow into the patient. In general, needle 130 is understood and appreciated to be a metal needle, but other materials may be used to provide the needle 130 without departing from the scope of the present invention.

In common everyday practice, the terms “needle” and “needle set” are often used interchangeably—for example a party may speak of an RMS HIgH-Fo™ “needle set” as simply RMS HIgH-Fo™ needles. But this is incorrect, for there is more involved than simply the sharp, hollow needle affixed to the distal end of the needle set assembly.

With respect to the present invention of HFLPIS 100, it is understood and appreciated that the advantages herein described are achieved as a result of the combination of at least the flexible tubing element 102 and the physical needle 130 and their various flow rate characteristics when combined advantageously.

RMS Medical Products of Chester, N.Y. is and has been a pioneer in needle set technology and flow rate control by means of specifically engineered flow control tubing. Indeed, RMS has realized that different flow rates may be provided by working with different flow combinations of flow control tubing, such as those systems and methods set forth in U.S. application Ser. No. 14/768,189 published as US 2015/0374911 entitled MULTI-FLOW UNIVERSAL TUBING SET, incorporated herein by reference, and U.S. Ser. No. 15/052,727 published as US 2016/0256625 entitled PRECISION VARIABLE FLOW RATE INFUSIONSYSTEM AND METHOD, incorporated herein by reference.

In sharp departure from the prevailing view within the infusion area of the medical community, RMS has advantageously developed the HFLPIS 100, wherein both the flexible tubing element 102 and the needle 130 are cooperatively combined to provide an advantageous high flow rate with a small needle at a low pressure. Indeed, it is an error to view the physical needle itself as the sole limiting factor.

With respect to FIG. 1, and more specifically FIG. 1A, and HFLPIS 100 it will be appreciated that the flexible tubing element 102 has a first length 104, and a first end, or inlet 106 structured and arranged to connect to a reservoir, not shown, such as by providing a connector 112, i.e. flared luer 112, and a second end 108 opposite to the first end 106, that is joined with the needle 130.

It is to be understood and appreciated that the flexible tubing element 102 is not general medical tubing. Although a tube by its very nature of being a tube may impart some element of flow restriction based on the size and length of the tube, general medical tubing has such a substantial internal diameter that any contribution of flow rate reduction is effectively negligible when dealing with liquids having a maximum dosage flow rate.

In contrast, flexible tubing element 102 has been specifically manufactured to have a specific length 104 and an internal diameter 110 (see FIG. 1B) so as to achieve a very specific, known and pre-defined flow rate for the flexible tubing element 102. Moreover, for at least one embodiment, the internal diameter 110 is substantially constant over the length of the flexible tubing element 102 from about the first end 106 to about the second end 108. Flexible tubing element 102 may also be referred to as flexible flow rate tubing flexible flow control tubing, or flow rate control tubing.

For at least one embodiment, the flexible tubing element 102 is 24-gauge tubing having an internal diameter 110 of about 0.32″-0.35″ and a length 104 of about 23.75″-24.24″.

The needle 130 is appreciated to have an internal diameter 132 and an outside diameter 134 and a length 136. The needle 130 has a first portion 138 providing a sharpened distal end 140 for penetration of a patient's tissues, and a second portion 142 providing a second end 144 in fluid communication with the second end 108 of the flexible tubing element 102.

It is to be noted that the outside diameter 134 of the needle is significantly smaller than the internal diameter 110 of the flexible tubing element 102, see FIGS. 1B and 1C. This is in sharp contrast to the traditional configuration of needle sets, wherein the outside diameter of the needle is substantially similar to the internal diameter of the tubing element, thus permitting ease of assembly.

For at least one embodiment, the needle 130 is a metal needle having a generally consistent thickness of material defining the internal diameter 132 and the outside diameter 134. As such, for at least one embodiment, it will be understood and appreciated that comparative relationships may be appreciated between the internal diameter 132 of the needle 130 and the average internal diameter 110 of the flexible tubing element 102, and the outside diameter 134 of the needle 130 and the average internal diameter 110 of the flexible tubing. More specifically, the internal diameter 132 and outside diameter 134 of the needle 130 are each substantially smaller than the average internal diameter 110 of the flexible tubing element 102.

Moreover, for at least one embodiment the average internal diameter 110 along the length of the flexible tubing element 102 is at least 10% larger than the internal diameter 132 of needle 130 extending from the second end 108. For yet another embodiment the average internal diameter 110 along the length of the flexible tubing element 102 is at least 25% larger than the internal diameter 132 of needle 130 extending from the second end 108. For yet another embodiment the average internal diameter 110 along the length of the flexible tubing element 102 is at least 50% larger than the internal diameter 132 of needle 130 extending from the second end 108.

Further, for at least one embodiment the average internal diameter 110 along the length of the flexible tubing element 102 is at least 10% larger than the outside diameter 134 of needle 130 extending from the second end 108. For yet another embodiment the average internal diameter 110 along the length of the flexible tubing element 102 is at least 25% larger than the outside diameter 134 of needle 130 extending from the second end 108. For yet another embodiment the average internal diameter 110 along the length of the flexible tubing element 102 is at least 50% larger than the outside diameter 134 of needle 130 extending from the second end 108.

More specifically, the relative size difference between the outside diameter 134 of the needle 130 and the internal diameter 110 of the flexible tubing element 102 presents a greater issue in manufacturing, and is likely at least a partial reason why this combination of “small needle” and “large tubing element” has heretofore not been readily available or even considered within the industry. Moreover, the outside diameter 134 of the needle is so substantially smaller than the internal diameter 110 of the flexible tubing element 102 that a traditional slip fit and glue assembly is inapplicable.

To achieve the second end 144 of the needle 130 in fluid communication with the second end 108 of the flexible tubing element 102, for at least one embodiment, the second end 108 of the flexible tubing element is necked down to adapt the larger internal diameter of the flexible tubing element 102 to the substantially smaller outside diameter 134 of the needle 130. This neck down, or necking down process may be performed in several ways without departing from the scope of the present invention.

For at least one embodiment, the neck down is achieved by compressing the second end 108 of the flexible tubing element 102 under heat to a smaller internal diameter to better hold the needle. This compressed end may then be sealed/bonded to the needle 130 with an adhesive. For yet another embodiment, at least one intermediate element 146 may be disposed between needle 130 and the inside of the flexible tubing element 102, such as, but not limited to, a ring, cylinder, strips, spacers, glue or other such material. And for still yet another embodiment, a combination of compressing the second end 108 and disposing at least one intermediate element 146 may be utilized.

As with the flexible tubing element 102, the needle 130 has a known length 136, the consistent internal diameter 132 in combination with the length providing a known flow rate for the needle 130. More specifically, as the length of the tube, or bore, through the needle 130 is a factor as well as the internal diameter of that tube, or bore, a short needle 130 is of significant importance for HFLPIS 100.

Moreover, to summarize, for at least one embodiment, provided is HFLPIS 100, including: a flexible tubing element 102 having a first length 104 and a first end 106 structured and arranged to connect to the reservoir, and a second end 108 opposite thereto, the tubing element having a first internal diameter 110; a needle 130 having a second length 136 and a second internal diameter 132, the needle 130 having a first portion 138 providing a sharpened distal end 140 for penetration of the patient's tissue and a second portion 142 providing a second end 144 in fluid communication with the second end 108 of the flexible tubing element 102; wherein the second end 144 of the needle 130 has an outside diameter 134, the flexible tubing element having an average first internal diameter 110 along the first length 104, the average first internal diameter 110 at least 25% larger than the outside diameter 134 of the second end of the needle 130.

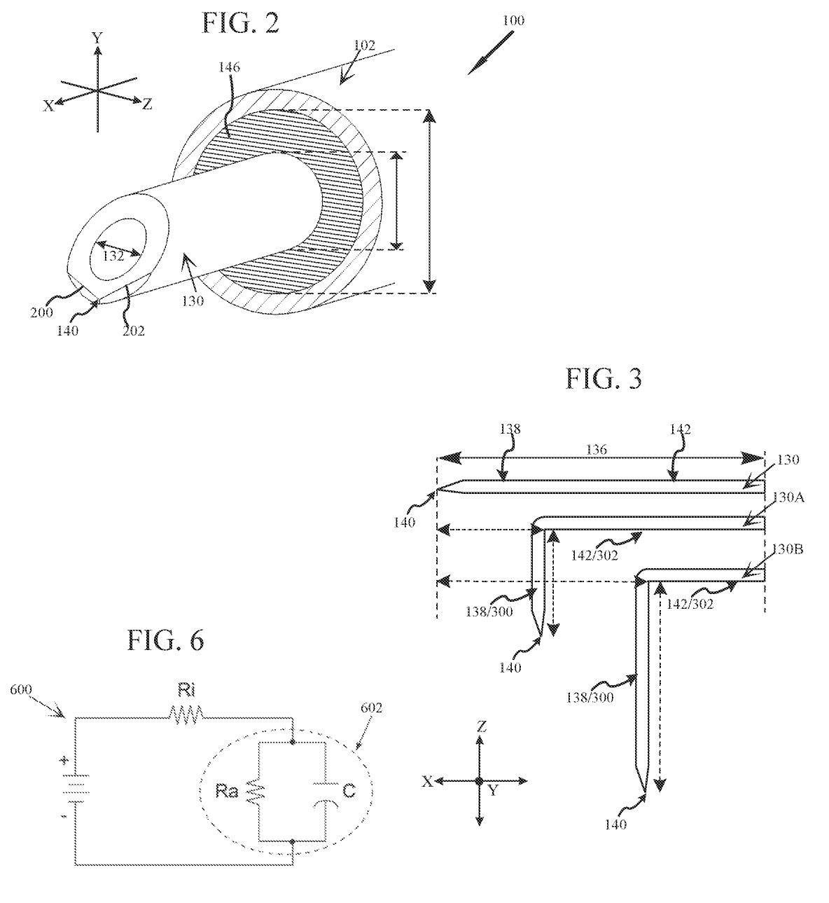

For at least one embodiment the needle 130 is a tricuspid needle 130, which may be more fully appreciated in FIG. 2. The tricuspid needle provides a greater cross-sectional area of flow.

As is clearly shown in the perspective view of FIG. 2, the tricuspid needle also provides two sharp edges, 200 and 202 which serve as cutting edges to ease passage of the needle 130 into and through the tissues of the patient by cutting the tissue along edges 200 and 202, as opposed to the more traditional needle with a single point that pushes/stretches the tissues out of the way.

Moreover, a typical needle with a sharp point, but no cutting edges punctures tissue and then forces tissue out of the way, thus causing stretching, distorting and/or tearing of the tissue, whereas the tricuspid needle with sharp edges, 200 and 202 cuts through tissues much as a scalpel, thus substantially avoiding the stretching, distorting and/or tearing of tissue.

FIG. 2 further illustrates an embodiment wherein an intermediate element 146 is disposed between the outside diameter 134 of the needle 130 and the inside diameter 110 of second end 108 of the flexible tubing element 102 so as to affix the needle 130 and flexible tubing element 102 together and in fluid communication.

For infusion purposes, it is generally important that the needle 130 be selected to penetrate to a specific depth. To facilitate this, the needle 130 often has a base which is intended to make direct contact with the patient's skin—this contact thus insuring that the depth of the needle 130 selected is correct. Moreover, the needle 130 may be a straight needle extending away from a base.

In many instances, the needle 130 itself is incorporated as part of this base. More specifically, as shown in FIG. 3, for at least one embodiment the needle 130 is bent to about a 90-degree angle, to provide a first portion 300 for coupling to the flexible tubing element 102 and a second portion 302 to be disposed into the patient.

In other words, for at least one embodiment the needle 130 has a first portion 300 providing a sharpened distal end for penetration of the patient's tissue and a second portion 302 providing a second end in fluid communication with flexible tubing element 102, the first portion 300 and second portion 302 generally normal to each other.

As the length 136 of the needle 130 in relation to the internal diameter is a factor in determining flow rate as noted above, to provide different needle 130 of different effective penetration lengths such as, but not limited to 4 mm, 6 mm, 9 mm, 12 mm, 14 mm and 16 mm, it will be understood that the length of the entire needle 130 may be constant—rather it is where the bend between the first portion 300 and the second portion 302 is disposed that helps determine the length of the second portion 302 and its associated penetration length.

With respect to FIG. 3, needles 130, 130A and 130B are shown—all having effectively the same length 136, with needles 130A and 130B bent to about a 90-degree angle, the first portion 300 of needle 130A being shorter than the first portion 300 of needle 130B, needles 130A and 130B thus being understood to correspond to different penetration lengths.

Moreover, the identification as a “short needle” is intended to help clarify for those in the infusion field that this needle 130 is indeed shorter than the general insulin needle administration sets wherein the needle elements are generally 2″ or more in length.

For at least one embodiment, the needle 130 is 26-gauge needle having an internal diameter 132 of about 0.0094″-0.0102″ and a length 136 of about 0.938″-0.962″.

As noted above, for at least one embodiment, the inlet 106 of the flexible tubing element 102 provides a connector 112 such as a luer 112, and for at least one embodiment, a flared luer 112. The luer 112 or flared luer 112 permits the inlet to be removably coupled to a reservoir, such as a syringe that is providing the pharmaceutical which will be passed through HFLPIS 100 and into the patient.

For some embodiments, an additional tubing element may be disposed between HFLPIS 100 and the reservoir, such as to permit greater distance between the reservoir and the patient. In other embodiments, an extra tubing element may not be employed, and the inlet 106 is received by a specific pump system, such as, but not limited to the Freedom60® Syringe Infusion Pump. For such embodiments, it is further understood and appreciated that the luer 112 of the inlet 106 is structured and arranged to receive the tip of a syringe, the syringe being the reservoir providing liquid.

The use of a flared luer 112 advantageously ensures that HFLPIS 100 is only used with pumps or other devices that have a corresponding base to receive the flared luer 112. The inlet 106 as a flared luer 112 is achieved in accordance with the systems and methods as set forth in U.S. Patent Application 62/274,487 and non-provisional U.S. patent application Ser. No. 15/291,895 claiming priority thereto, each entitled “SYSTEM AND METHOD FOR FLARED LUER CONNECTOR FOR MEDICAL TUBING” and each incorporated herein by reference.

With respect to the specific and advantageous nature of the flexible tubing element 102 having a known and specific length 104 and a known and substantially consistent internal diameter 110, and the needle having a known and specific length 136 and a substantially consistent internal diameter 132, it will be appreciated that the needle 130 and the flexible tubing element 102 collectively interact to provide an overall known flow rate.

Flow rate through a tube is generally predicted by, Equation #1:

$$Q = \frac{Δ P}{R} \qquad Equation \#1$$

where: Q=flow rate;

ΔP=pressure (differential over the length of the tube);

R=the resistance faced by the fluid that is flowing.

The flexible tubing element 102 is specifically developed to provide a laminar flow, also known as a streamline flow. Laminar flow occurs when a fluid flows in parallel layers, with no disruption between the layers. At low velocities, the fluid tends to flow without lateral mixing, which means that the adjacent layers slide past one another. This lack of mixing between layers means that there are no cross-currents, eddies or swirls of the fluid—the motion of the particles of the fluid is very ordinary with all particles moving in a straight line relative to the side walls of the flexible flow rate tubing.

With respect to fluid dynamics, the Reynolds number is an important parameter in equations that describe whether fully developed flow conditions lead to laminar or turbulent flow. The Reynolds number is the ratio of the internal force to the shearing force of the fluid—in other words, how fast the fluid is moving relative to how viscous the fluid is, irrespective of the scale of the fluid system. Laminar flow generally occurs when the fluid is moving slowly or the fluid is very viscous.

The specific calculation of the Reynolds number and the values where laminar flow occurs depends on the geometry of the flow system and flow pattern, in this case primarily the flexible tubing, which parallels the common example of flow through a pipe, where the Reynolds number is defined as shown by Equation #2:

$$Re = \frac{ρ {vD}_{H}}{μ} = \frac{{vD}_{H}}{v} = \frac{{QD}_{H}}{vA} \qquad Equation \#2$$

where: D_{H} is the hydraulic diameter of the pipe (flexible tubing element 102); its characteristic traveled length, L, (m).

Q is the volumetric flow rate (m³/s).

A is the pipe cross-sectional area (m²) of the pipe (flexible tubing element 102).

V is the mean velocity of the fluid (SI units: m/s).

μ is the dynamic viscosity of the fluid (Pa·s=N·s/m²=kg/(m·s)).

V is the kinematic viscosity of the fluid (V=μ/ρ) (m²/s).

ρ is the density of the fluid (kg/m³).

Moreover, flexible tubing element 102 is designed with specific characteristics in light of the above Reynolds equation so as to provide an environment conducive to Laminar flow of intended fluids for use with HFLPIS 100. In other words, those skilled in the art will appreciate that flexible tubing element 102 is formed with a specific length and consistent internal diameter so as to achieve an environment conducive to Laminar flow.”

Although a low flow rate may be directed through general medical tubing, the low flow rate is achieved by means other than the general tubing, as general tubing does not impart a significant element of flow rate control. When and as the flow rate increases through the general medical tubing, more often than not the flow rate becomes transient, also known as unsteady, or even turbulent. In either case, the flow rate is not consistent and may be problematic.

With respect to HFLPIS 100 by being structured and arranged to provide a laminar flow, flexible tubing element 102 is able to impart and maintain a consistent pre-determined flow rate, which as is further described below, is highly advantageous to HFLPIS 100. With respect to HFLPIS 100 and more specifically flexible tubing element 102, laminar flow is defined as fluid flow with Reynolds numbers less than 2300. Of course, it is understood and appreciated that transition and turbulent flow can, however, be observed below 2300 in some situations.

The nature of the flexible tubing element 102 to advantageously provide laminar flow, is further enhanced in situations where the liquid being infused to the patient is a Newtonian fluid. A Newtonian fluid is a fluid in which the viscous stresses arising from its flow are linearly proportional to the local strain rate, which is the rate of change of deformation over time. Water, organic solvents and honey are some examples of Newtonian fluids where viscosity remains constant no matter the amount of shear applied for a constant temperature. As infusion treatments generally are intended to provide the patient with a specific medication or composition, many of the fluids desired for use with HFLPIS 100 are Newtonian fluids. As such, the ability of HFLPIS 100 to provide fine grain flow control is further enhanced.

Having introduced the principles for laminar flow above, it is further appreciated that for the entire needle set system as provided by HFLPIS 100, predicted flow rate should be based not just on the needle, but on the entire needle set system.

Moreover, predicted flow rate is determined by the Hagen Poiseuille equation, shown as Equation #3:

$$Q = \frac{π {r}^{4} Δ P}{8 μ L} \qquad Equation \#3$$

where: Q=flow rate;

r=radius of the tube;

ΔP=pressure (differential over the length of the tube);

μ=viscosity; and

L=length.

This equation shows that the predicted fluid flow rate is directly proportional to the difference in the pressure from inlet to outlet and the fourth power of the diameter, inversely proportional to the viscosity and length of the flexible tubing element 102.

$$Re = \frac{V ρ ID}{μ} \qquad Equation \#4$$

where: V=the mean velocity of the fluid flowing through the cylinder;

ρ=the density of the fluid;

ID=inner diameter of the cylinder;

μ=viscosity of the fluid.

$$V = \frac{Q}{A} = Q \text{/} ( π * {( \frac{ID}{2} )}^{2} ) \\ Q = \frac{π * Δ P * {r}^{4}}{8 * L * μ} \\ Re = \frac{Δ {PID}^{3} ρ}{32 {μ}^{2} L} \qquad Equation \#5$$

Therefore, the Reynolds number is proportional to the cube of inner diameter. The present invention therefore has specifically reduced the inside diameter 110 of the flexible tubing element 102 to be well below general medical tubing so as to ensure laminar flow, as noted above.

The use of this equation for determining the fluid flow through a tube (pipe) depends on the fluid meeting the Newtonian assumption, specifically that the fluid stays in laminar flow (Reynolds number <2300), and the length is much longer than the diameter. If all of these assumptions are met, then flow rates of different elements can be calculated along the same lines as electric circuits.

Electric circuits can be calculated as elements or groups; the needle sets are one such sub-set of elements consisting of a smaller tube (the needle 130) and a longer bigger tubing connected to a luer connector (flexible tubing element 102).

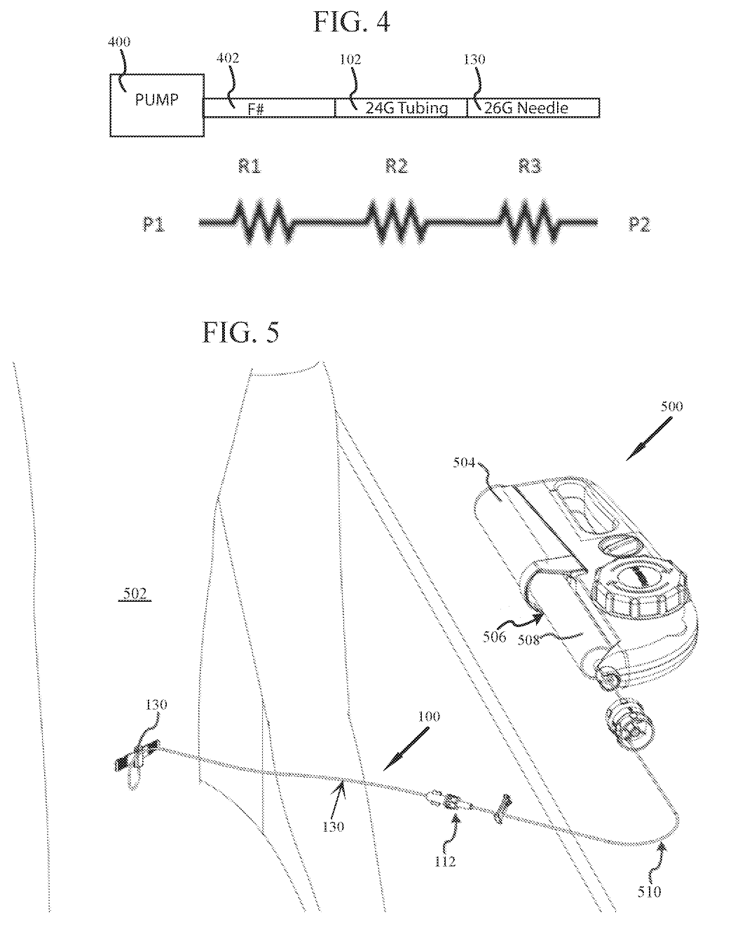

This may be more fully appreciated by a review of the following example in connection with FIG. 4 showing a conceptualized model of an infusion system using HFLPIS 100. As shown in FIG. 4, the infusion system is initiated by a pump 400 which will provide pressure to drive the pharmaceutical through the system. As noted above, in varying embodiments, HFLPIS 100 may be connected to a traditional tubing element 402 which does not impose a significant flow rate to the pharmaceutical—the purpose of this traditional tubing element being to permit convenient placement of the pump in one location and comfortable placement and position of the receiving patient in a second location.

The traditional tubing element 402 is coupled to the HFLPIS 100, which for this exemplary embodiment incorporates a 24-gauge tubing element as the flexible tubing element 102, and a 26-gauge needle 130. Once flow (Q) is calculated for each component, the total flow (Qₜₒₜₐₗ) is calculated as shown by Equation #4:

$${Q}_{total} = \frac{Δ P}{{R}_{total}} = \frac{Δ P}{\frac{Δ P}{\frac{1}{{Q}_{F}} + \frac{1}{{Q}_{T}} + \frac{1}{{Q}_{N}}}} = \frac{1}{\frac{1}{{Q}_{F}} + \frac{1}{{Q}_{T}} + \frac{1}{{Q}_{N}}} \qquad Equation \#4$$

For the exemplary calculations that follow, the following are assumed:
The minimum, normal and maximum back pressure for the pump 300 are 13.3 PSI, 13.5 PSI, and 13.7 PSI respectively.
The viscosity of water is 1 mPa s
Inner Diameter of the 26-gauge needle is 0.0094″-0.0102″
Length of the 26-gauge needle is 0.938″-0.962″
Inner diameter of 24-gauge tubing is 0.032″-0.035″
Length of the 24-gauge tubing is 23.75″-24.24″

Pressure is noted for this example to be as follows:

P₁=13.5 PSI, P₂=0 (ATM), ΔP=13.5 PSI

The resistance of the components—traditional tubing element 302, the flexible tubing element 102 and the needle 130, and flow rates (Q) are determined as follows:

$${R}_{1} = \frac{Δ P}{{Q}_{F}} = \frac{13.5 PSI}{F \#}$$

$${R}_{2} = \frac{13.5 PSI}{24 G Raw Tubing flow rate}$$

$${R}_{3} = \frac{13.5 PSI}{26 G Needle flow rate}$$

$${R}_{total} = {R}_{1} + {R}_{2} + {R}_{3}$$

$${Q}_{total} = \frac{Δ P}{{R}_{total}} = \frac{Δ P}{\frac{Δ P}{\frac{1}{{Q}_{F}} + \frac{1}{{Q}_{T}} + \frac{1}{{Q}_{N}}}} = \frac{1}{\frac{1}{{Q}_{F}} + \frac{1}{{Q}_{T}} + \frac{1}{{Q}_{N}}}$$

$${Q}_{S} = Super 26 Total Flow rate = \frac{{Q}_{T} {Q}_{N}}{{Q}_{T} + {Q}_{N}}$$

$${Q}_{total} = \frac{{Q}_{F} {Q}_{S}}{{Q}_{F} + {Q}_{S}}$$

For comparison, both the minimum and maximum flow values are shown based on the minimum and maximum dimensions for the flexible tubing element 102 and needle 130 as noted above. First the minimum total flow.

$${Q}_{S 26 @ 13 PSI} = \frac{{Q}_{Tmin} {Q}_{Nmin}}{{Q}_{Tmin} + {Q}_{Nmin}}$$

HP equation(Equation #3 above):

$$Q = \frac{πΔ {P ( r )}^{4}}{8 μ L}$$

$${Q}_{N 26 min} = \frac{π13 .3 {PSI ( \frac{{ID}_{min}}{2} )}^{4}}{8 ( 1 cP ) Lmax}$$

$${Q}_{N 26 min} = \frac{π13 .3 {PSI ( \frac{{.0094}^{″}}{2} )}^{4}}{8 ( 1 cP ) {.962}^{″}}$$

$${Q}_{N 26 min} = ∼ 1078 ml \text{/} hr$$

$${Q}_{T 24 min} = \frac{π13 .3 {PSI ( \frac{{ID}_{min}}{2} )}^{4}}{8 ( 1 cP ) Lmax}$$

$${Q}_{T 24 min} = \frac{π13 {PSI ( \frac{{.032}^{″}}{2} )}^{4}}{8 ( 1 cP ) {24.25}^{″}}$$

$${Q}_{T 24 min} = 5741 ml \text{/} hr$$

$${Q}_{S 26 @ 13.3 PSI} = \frac{5741 ( 1078 )}{5741 + 1078} \frac{ml}{hr} = ∼ 907 ml \text{/} hr$$

Now the maximum total flow.

$${Q}_{S 26 @ 13 PSI} = \frac{{Q}_{Tmax} {Q}_{Nmax}}{{Q}_{Tmax} + {Q}_{Nmax}}$$

HP equation(Equation #3 above):

$$= \frac{πΔ {P ( r )}^{4}}{8 μ L}$$

$${Q}_{N 26 max} = \frac{π13 .3 {PSI ( \frac{{ID}_{max}}{2} )}^{4}}{8 ( 1 cP ) Lmin}$$

$${Q}_{N 26 max} = \frac{π13 .3 {PSI ( \frac{{.0102}^{″}}{2} )}^{4}}{8 ( 1 cP ) {.938}^{″}}$$

$${Q}_{N 26 max} = ∼ 1600 ml \text{/} hr$$

$${Q}_{T 24 max} = \frac{π13 .3 {PSI ( \frac{{ID}_{max}}{2} )}^{4}}{8 ( 1 cP ) Lmin}$$

$${Q}_{T 24 max} = \frac{π13 {PSI ( \frac{{.035}^{″}}{2} )}^{4}}{8 ( 1 cP ) {23.75}^{″}}$$

$${Q}_{T 24 max} = ∼ 8500 ml \text{/} hr$$

$${Q}_{S 26 max @ 13.3 PSI} = \frac{8850 ( 1600 )}{8850 + 1600} \frac{ml}{hr} = ∼ 1360 ml \text{/} hr$$

Moreover, the apparent range of flow rate permitted by this configuration is ˜907 ml/hr to =˜1360 ml/hr.

For the sake of further comparison and appreciation of the advantageous nature of HFLPIS 100, the same calculations are performed with respect to the RMS HigHFlo 26 needle set—which comprises a 26-gauge needle with 26-gauge tubing.

$${Q}_{26 G @ 13 PSI} = \frac{{Q}_{Tmax} {Q}_{Nmax}}{{Q}_{Tmax} + {Q}_{Nmax}}$$

$${Q}_{N 26 min} = \frac{π13 .3 {PSI ( \frac{{ID}_{min}}{2} )}^{4}}{8 ( 1 cP ) Lmax} = ∼ 1100 ml \text{/} hr$$

$${Q}_{T 26 min} = \frac{π13 .3 {PSI ( \frac{{ID}_{min}}{2} )}^{4}}{8 ( 1 cP ) Lmax} = ∼ 860 ml \text{/} hr$$

$${Q}_{26 Gmin @ 13.3 PSI} = \frac{1100 ( 860 )}{110 + 860} \frac{ml}{hr} = ∼ 480 ml \text{/} hr$$

Moreover, with respect to the above calculations, it will be understood and appreciated that for at least one embodiment of the HFLPIS 100, such as the RMS Super 26 needle set, under ideal conditions the flow will be 2.8 times faster than the regular HIgHFlo 26G needle set.

Of course, it is understood and appreciated that these flow rates may be throttled back to the prescribed flow rate intended by the manufacturer or doctor for the type of infusion to be performed. Indeed, in varying embodiments, traditional tubing element 402 may be coupled to or replaced by a flow control tubing system as presented by the above noted application Ser. No. 14/768,189, which is in turn coupled to HFLPIS 100.

This result is highly advantageous and confirms that embodiments of HFLPIS 100 can indeed provide high flow rates at low pressures. More specifically, in sharp contrast to the assumed norm based on the misconception that the needle at the end of the tubing is the final component of flow rate, as the above material so demonstrates, if the fluid flow remains in a laminar state, then the combining equations may be safely used to predict the flow rate.

If the fluid flow begins to diverge from laminar, there will still be some increased flow rate with increasing pressures, but the relationship will diverge from linear until at some very high pressure, there will be very little increase in flow rate with increasing pressure, but that usually occurs at pressures far in excess of the levels used for infusions when HFLPIS is used with intended infusion systems such as the RMS Freedom system.

For at least one embodiment, as shown in FIG. 5, HFLPIS 100 is incorporated as part of an infusion system 500 for delivery of a pharmaceutical to a patient 502. Moreover, for at least one embodiment HFLPIS 100 is intended for use with a constant pressure pump 504, such as the Freedom60® Syringe Infusion Pump as provided by RMS Medical Products of Chester, N.Y. Constant pressure systems, such as the Freedome60®, when combined with HFLPIS 100 may be highly advantageous in preventing unintended and/or unsafe rates of administration of the liquid to the patient.

For the conceptual infusion therapy session depicted by FIG. 5, a reservoir 506 is disposed within pump 504, the reservoir providing a liquid 508, such as a pharmaceutical. The outlet of the reservoir 506 is coupled to a first tubing 510, which has been depicted as a flow controlling tubing element consistent with US 2016/0256625 as noted above, though normal non-flow regulating tubing may also be used. This tubing—if used, is then coupled to HFLPIS 100, the needle 130 of which is disposed into the patient 502. Of course, in varying embodiments, the first tubing 510 may be entirely omitted and HFLPIS 100 may be connected directly to the outlet of the reservoir 506.

With a constant flow rate system, the pressure is increased in response to any flow restriction no matter if such a restriction is the buildup of pressure in the patient's tissues or an element of the delivery system. This can result in an administration of the liquid at an unsafe pressure. As such, for an intravenous administration, the patient may suffer a wide range of symptoms, including, but not limited to, infiltration, extravasation, vein collapse, anaphylaxis, overdose, histamine reactions, morbidity, and mortality. For subcutaneous administrations for which the HFLPIS 100 is intended, the effects of unsafe pressures result in site reactions, such as pain, swelling, redness, itching, leakage, and general discomfort.

In sharp contrast, with a constant pressure rate system, such as the Freedome60®, if there is a pinch in the tubing, blockage in the infusion system or blockage in the patient's body (such as by saturation of the tissues for SQ or a vein collapse for IV), such an event results in resistance to the flow and affects the flow rate, not the pressure, i.e., the flow rate decreases as the pressure increases. A constant pressure system may be compared to a theoretical model of an electrical system shown in FIG. 6.

For the exemplary electrical system 600, as resistance increases 602, the current will immediately and proportionally decrease. A constant pressure infusion system produces this same result: if the resistance to flow increases, the system will immediately adjust by lowering the flow rate. This insures—by design—that a patient can never be exposed to a critically high pressure of liquid.

Moreover, as HFLPIS 100 establishes an upper boundary for flow rate of a liquid from a reservoir at or below a pre-defined flow rate, embodiments of HFLPIS 100 are suitable for infusion treatments with constant pressure systems. Additional advantages may be provided when embodiments of HFLPIS 100 are combined with a constant pressure pump such as the Freedom60®.

With respect to use with a constant rate or constant flow electric pump, HFLPIS 100 is also advantageous over existing options. More specifically, the low resistance of the HFLPIS 100 will keep pressure lower, preventing damage to some pumps from excessive high pressure to maintain a flow rate, and from potentially unnecessary alarms which might shut down the administration and inconveniencing the patient or care giver by requiring a system reset, and/or delaying the needed medication at the time of infusion. Moreover, HFLPIS 100 does not and should not be perceived as a solution to the potential danger presented by constant rate or constant flow electric pumps—but it may help reduce the chance and/or frequency of such risks.

Having described embodiments of HFLPIS 100, other embodiments relating to at least one method of using HFLPIS 100 will now be discussed. It will be appreciated that the described method need not be performed in the order in which it is herein described, but that this is merely exemplary of one method of using HFLPIS 100.

In general, for at least one embodiment, the method commences with coupling a reservoir containing a liquid to a pump for driving the liquid from the reservoir. An embodiment of HFLPIS 100, as described above, is then provided as well. HFLPIS 100 is coupled to the reservoir and the needle is disposed into the patient. With activation of the pump, HFLPIS 100 adventitiously permits the infusion to occur with high flow at low pressure, with a smaller needle 130 this is otherwise permitted with traditional infusion needle sets.

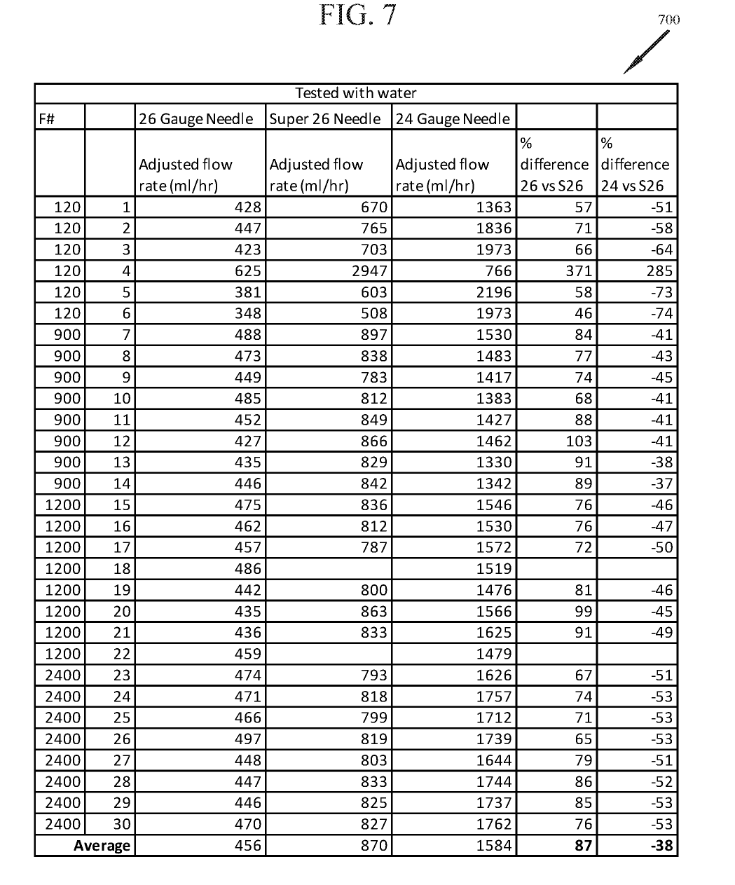

Testing of embodiments of the present invention for HFLPIS 100 has demonstrated the advantages of HFLPIS 100. A selection of this test data is presented in FIG. 7 as table 700. The data presented in table 700 was acquired through the following testing procedture.

Eight units of RMS 24-gauge needle subassemblies, RMS 26-gauge needle subassemblies, and RMS Super26 needle subassemblies (an embodiment of HFLPIS 100), respectively were connected to eight units of RMS F120, F900, F1200 and F2400 tubing. Each type of unit was sourced from three separate lots each. The combination of needle and tubing was set in line with a 60 ml syringe filled with water and pressurized to 13.5 PSI, to simulate a Freedom 60 pump, tubing and needle set up.

The flow was collected in a beaker set on a balance that recorded the weight at the start of the measurement and at the end of the measurement. This mass flow rate was converted into a volumetric flow rate by dividing by the density of water at the temperature of the test fluid measured at the start of the test. The flow rate of the individual pieces of 24-gauge needle, 26-gauge needle and Super 26 was calculated. These calculated values are displayed in table 700. The percent increase of the flow rate between the Super26 and the 26-gauge is listed in the second to last row. The percent decrease in flow rate between the Super26 and the 24-gauge needle is listed in the last column. The average flow rates, and averages are listed in the final row. It is noteworthy that the Super26 achieves almost a 90% increase in flow rate compared to the 26-gauge needle set in initial testing. This demonstrates the large advantage to the novel step of drastically increasing the inner diameter of the tubing that leads to the 26-gauge needle in the super 26 needle subassembly, e.g., an embodiment of HFLPIS 100.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A high flow at low pressure infusion system needle set for delivering a liquid from a reservoir to a patient, comprising:
a flexible tubing element having a first length and a first end structured and arranged to connect to the reservoir, and a second end opposite thereto, the flexible tubing element having a first internal diameter;
a needle having a second length and a second internal diameter, the needle having a first portion providing a sharpened distal end for penetration of the patient's tissue and a second portion providing a second end in fluid communication with the second end of the flexible tubing element;
wherein the second end of the needle has an outside diameter, the flexible tubing element having an average first internal diameter along the first length, the average first internal diameter at least 25% larger than the outside diameter of the second end of the needle.

2. The high flow at low pressure infusion system needle set of claim 1, wherein for a viscus fluid in a laminar state flowing from the reservoir to the patient through the flexible tubing element and the needle, the flexible tubing element and the needle permit a rapid flow rate of between about 50 ml/hr and 90 ml/hr.

3. The high flow at low pressure infusion system needle set of claim 2, wherein the flow rate is at least 100 ml/hr.

4. The high flow at low pressure infusion system needle set of claim 1, wherein the average first internal diameter is at least 50% larger than the outside diameter of the second end of the needle.

5. The high flow at low pressure infusion system needle set of claim 1, wherein the first length of the flexible tubing element is about 24″.

6. The high flow at low pressure infusion system needle set of claim 1, wherein the needle has a maximum second length of about 0.95″.

7. The high flow at low pressure infusion system needle set of claim 1, wherein the needle is a thin wall 26-gauge needle.

8. The high flow at low pressure infusion system needle set of claim 1, wherein the needle has an internal diameter of about 0.0094″.

9. The high flow at low pressure infusion system needle set of claim 1, wherein the second end of the flexible tubing element is bonded to the second end of the needle.

10. The high flow at low pressure infusion system needle set of claim 1, wherein the second end of the flexible tubing element is necked downed to about an outside diameter of the second end of needle.

11. The high flow at low pressure infusion system needle set of claim 1, wherein a neck down element is disposed between the second end of the flexible tubing element and the second end of the needle.

12. The high flow at low pressure infusion system needle set of claim 1, wherein the liquid is dispensed from the reservoir into the needle set by a fluid pump.

13. The high flow at low pressure infusion system needle set of claim 12, wherein the fluid pump is a constant force pressure pump.

14. The high flow at low pressure infusion system needle set of claim 12, wherein the pressure provided by the fluid pump does not exceed about 14 psi.

15. The high flow at low pressure infusion system needle set of claim 1, wherein the first end of the flexible tubing element includes a flared luer.

16. The high flow at low pressure infusion system needle set of claim 1, wherein the needle is a tricuspid needle.

17. The high flow at low pressure infusion system needle set of claim 1, wherein the first portion and second portion generally normal to each other

18. A high flow at low pressure infusion system needle set for delivering a liquid from a reservoir to a patient, comprising:
a flexible tubing element having a first length of about 24″ and a first end structured and arranged to connect to the reservoir, and a second end opposite thereto, the flexible tubing element having a first internal diameter of at least 0.33″;
a needle having a maximum second length of about 0.95″ and a second internal diameter of about 0.0094″, the needle having a first portion providing a sharpened distal end for penetration of the patient's tissue and a second portion providing a second end in fluid communication with the second end of the flexible tubing element, the first portion and second portion generally normal to each other;
wherein the second end of the needle has an outside diameter, the flexible tubing element having an average first internal diameter along the first length, the average first internal diameter at least 25% larger than the outside diameter of the second end of the needle.

19. The high flow at low pressure infusion system needle set of claim 18, wherein for a viscus fluid in a laminar state flowing from the reservoir to the patient through the flexible tubing element and the needle, the flexible tubing element and the needle permit a rapid flow rate of between about 50 ml/hr and 90 ml/hr.

20. The high flow at low pressure infusion system needle set of claim 18, wherein the needle is a thin wall 26-gauge needle.

21. The high flow at low pressure infusion system needle set of claim 18, wherein the second end of the flexible tubing element is bonded to the second end of the needle.

22. The high flow at low pressure infusion system needle set of claim 18, wherein the second end of the flexible tubing element is necked downed to about an outside diameter of the second end of needle.

23. The high flow at low pressure infusion system needle set of claim 18, wherein a neck down element is disposed between the second end of the flexible tubing element and the second end of the needle.

24. The high flow at low pressure infusion system needle set of claim 18, wherein the liquid is dispensed from the reservoir into the needle set by a fluid pump.

25. The high flow at low pressure infusion system needle set of claim 24, wherein the fluid pump is a constant force pressure pump.

26. The high flow at low pressure infusion system needle set of claim 24, wherein the pressure provided by the fluid pump does not about 14 psi.

27. The high flow at low pressure infusion system needle set of claim 18, wherein the first end of the flexible tubing element includes a flared luer.

28. The high flow at low pressure infusion system needle set of claim 18, wherein the needle is a tricuspid needle.

29. A high flow at low pressure infusion system for delivering a liquid from a reservoir to a patient, comprising:
a fluid pump for driving a fluid from the reservoir;
a flexible tubing element having a first length and a first end structured and arranged to connect to the reservoir, and a second end opposite thereto, the flexible tubing element having a first internal diameter selected with respect to the first length to provide laminar flow for a liquid having a known viscosity, received from the reservoir;
a needle having a second length and a second internal diameter selected to maximize flow rate to a patient's tissues at a specific depth, the needle having a first portion providing a sharpened distal end for penetration of the patient's tissue to the specific depth and a second portion providing a second end in fluid communication with the second end of the flexible tubing element, the first portion and second portion generally normal to each other;
wherein the second end of the needle has an outside diameter, the flexible tubing element having an average first internal diameter along the first length, the average first internal diameter at least 25% larger than the outside diameter of the second end of the needle.

30. The high flow at low pressure infusion system needle set of claim 29, wherein for a viscus fluid in a laminar state flowing from the reservoir to the patient through the flexible tubing element and the needle, the flexible tubing element and the needle permit a rapid flow rate of between about 50 ml/hr and 90 ml/hr.

31. The high flow at low pressure infusion system needle set of claim 29, wherein the first length of the flexible tubing element is about 24″.

32. The high flow at low pressure infusion system needle set of claim 29, wherein the needle has a maximum second length of about 0.95″.

33. The high flow at low pressure infusion system needle set of claim 29, wherein the needle is a thin wall 26-gauge needle.

34. The high flow at low pressure infusion system needle set of claim 29, wherein the needle has an internal diameter of about 0.0095″.

35. The high flow at low pressure infusion system needle set of claim 29, wherein the second end of the flexible tubing element is bonded to the second end of the needle.

36. The high flow at low pressure infusion system needle set of claim 29, wherein the second end of the flexible tubing element is necked downed to about an outside diameter of the second end of needle.

37. The high flow at low pressure infusion system needle set of claim 29, wherein a neck down element is disposed between the second end of the flexible tubing element and the second end of the needle.

38. The high flow at low pressure infusion system needle set of claim 29, wherein the fluid pump is a constant force pressure pump.

39. The high flow at low pressure infusion system needle set of claim 29, wherein the first end of the flexible tubing element includes a flared luer.

40. The high flow at low pressure infusion system needle set of claim 29, wherein the pressure provided by the fluid pump does not exceed about 14 psi.

41. The high flow at low pressure infusion system needle set of claim 29, wherein the needle is a tricuspid needle.

42. A method of using a high flow at low pressure infusion system needle set for delivering a liquid from a reservoir to a patient, comprising:
coupling a reservoir containing a liquid to a pump for driving the liquid from the reservoir;
providing a high flow at low pressure needle set including:
a flexible tubing element having a first length and a first end structured and arranged to connect to the reservoir, and a second end opposite thereto, the flexible tubing element having a first internal diameter;
a needle having a second length and a second internal diameter, the needle having a first portion providing a sharpened distal end for penetration of the patient's tissue and a second portion providing a second end in fluid communication with the second end of the flexible tubing element;
wherein the second end of the needle has an outside diameter, the flexible tubing element having an average first internal diameter along the first length, the average first internal diameter at least 25% larger than the outside diameter of the second end of the needle;
coupling the high flow at low pressure needle set to a reservoir and disposing the needle into a patient, and activating the pump.

43. The method of claim 42, wherein for a viscus fluid in a laminar state flowing from the reservoir to the patient through the flexible tubing element and the needle, the flexible tubing element and the needle permit a rapid flow rate of between about 50 ml/hr and 90 ml/hr.

44. The method of claim 42, wherein the needle is a thin wall 26-gauge needle.

45. The method of claim 42, wherein the fluid pump is a constant force pressure pump.

46. The method of claim 42, wherein the pressure provided by the fluid pump does not about 14 psi.

47. The method of claim 42, wherein the first end of the flexible tubing element includes a flared luer.

48. The method of claim 42, wherein the needle is a tricuspid needle.
